(19) United States
(12) Patent Application Publication
(10) Pub. No.: US 2014/0081256 A1
(43) Pub. Date: Mar. 20, 2014

(54) PORTABLE ELECTROSURGICAL INSTRUMENTS AND METHOD OF USING SAME

(71) Applicant: ElectroMedical Associates LLC, Bethesda, MD (US)

(72) Inventors: Yuval CARMEL, Rockville, MD (US); Anatoly Shkvarunets, Rockville, MD (US); Robert A. Van wyk, St. Pete Beach, FL (US)

(73) Assignee: ElectroMedical Associates LLC, Bethesda, MD (US)

(21) Appl. No.: 14/024,912

(22) Filed: Sep. 12, 2013

Related U.S. Application Data
(60) Provisional application No. 61/743,885, filed on Sep. 12, 2012.
(60) Provisional application No. 61/956,357, filed on Jun. 6, 2013.

(51) Int. Cl. A61B 18/18

(52) U.S. Cl. CPC A61B 18/18
USPC 606/33

(57) ABSTRACT

In contrast to instruments of the prior art that require bulky, cumbersome and/or costly electrical connections and energy sources, instruments designed in accordance with the instant disclosure are both portable and self-powered. The electrosurgical instruments of the present invention are not limited to a particular use or construction and can be adapted for operation with or without a patient return electrode (sometimes referred to as return pad or plate), in dry or wet fields, in the presence of bodily fluids (such as blood saliva and more), electrically conductive or non-conductive fluids. They may further be optionally equipped or configured for irrigation and or aspiration of liquids, gases or cryogenics, either external, remote or on-board. The electrode component of the electrosurgical instrument of the present invention may be monopolar, bipolar, or multipolar and may optionally include one or more floating electrodes. The electrosurgical instruments of the present invention may be single use (disposable) or multi-use (reusable) and can be compatible with various image-guiding systems, like fluoroscopic, ultrasound and others.

### PRIORITY

This application claims the benefit of U.S. Provisional Application Ser. Nos. 61/743,885 filed Sep. 12, 2012 and 61/956,357 filed Jun. 6, 2013, the entire contents of which are incorporated by reference herein.

### FIELD OF THE INVENTION

The present invention generally relates to instruments and systems for energy-based medical therapy. More specifically, the invention relates to portable, efficient electrosurgical instruments, both disposable and reusable, suitable for delivering both heat and energy into a soft tissue, that, in contrast to prior art devices, require no bulky, costly and/or dedicated energy sources.

### BACKGROUND OF THE INVENTION

The first reported medical application of electrosurgery dates back to October 1926, when Drs. Cushing and Bovie were able to satisfactorily remove a tumor from a patient's head with practically none of the bleeding associated with conventional procedures. Over the years, the popularity of electrosurgical procedures has grown dramatically due to impressive improvements in equipment, devices and surgical techniques. It is estimated that electrosurgical devices/systems are presently used in 80% of all surgical procedures performed.

Electrosurgery involves the application of high frequency energy to modify the structure or integrity of a subject tissue, more specifically, the application of high frequency energy sufficient to cut, coagulate, vaporize, seal, shrink, resect, excise, sculpt, ablate, denaturize and/or otherwise modify a target soft tissue in both human and veterinary subjects. For these reasons, electrosurgical systems play an important role in a wide variety of medical procedures including, but not limited to, cardiology, general surgery, ophthalmology, arthroscopy, urology, gynecology, laparoscopy, ear nose and throat, dermatology, GI, oncology, cosmetic and plastic surgery.

Presently available electrosurgical instruments for medical application tend to be energized by external, sophisticated, bulky and costly energy sources accessible through conventional electrical cords. Typically, the medical staff and/or the surgeons set such energy sources to a parameter setting suitable for a particular medical procedure. The selected parameter setting of these energy sources can vary greatly from one medical procedure to the next.

Despite their popularity, currently available electrosurgical instruments and systems are subject to important limitations and restrictions, examples of which include, but are not limited to:
(1) Restricted motion of the surgeon: The surgeon's motion in the operating room is limited by the length of the electrical cord(s) connecting the electrosurgical device(s) to the energy source(s) (e.g., a radiofrequency (RF) medical generator).
(2) Increased cost: An RF medical generator is an expensive, sophisticated dedicated piece of capital equipment that increases the overall cost of medical procedures. It requires service, cleaning, calibration and repair, all of which increase the usage cost and thus the overall cost for delivering medical care.
(3) Reduced patient safety due to setting errors: An RF medical generator is usually positioned in the operating room outside the field of view of the surgeon, thereby preventing him from monitoring its front panel setting (and thus the applied parameters) during use. Such a lack of oversight can lead to setting errors that adversely effect patient safety. This is a particularly significant concern with modern RF generators that have their own screens, receptacles and push buttons.
(4) Reduced reliability and safety due to electrical cords: The use of overlong electrical cords extending between instrument and power source can lead to electromagnetic interference (RFI) with other devices in the operating room, such as video monitors, computers, phones and pacemakers. However, shorter electrical cords can restrict the motion of the medical staff during the procedure. In either case, such cords can be inadvertently disconnected or damaged. Moreover, electrical energy is dissipated in the electrical cords, making it necessary to operate at energy levels higher than otherwise necessary.
(5) Limited ability to fully benefit from electrosurgical procedures: Some facilities like field hospitals, emergency rooms, veterinary clinics and doctor offices may not be adequately equipped with the requisite modern generators.

The present invention herein disclosed addresses these and other limitations.

Prior art portable medical instruments can be divided into two categories: (a) cauteries and (b) mechanical devices. The former are exemplified by single use, battery operated cauteries such as made by Bovie Medical (Clearwater, Fla.). Such devices use direct current (DC) from batteries to heat a filament. Devices in the latter category generally operate by producing mechanical motion or vibrations. Examples of such cordless surgical devices based on mechanical motion include battery operated drills and saws for bones and hard tissue, such as made for example by Arthrex ((Naples, Fla.), and a battery powered mechanical shaver (debrider) by Olympus (Southborough, Mass.). An example of a cordless surgical device based on mechanical vibrations includes the cordless ultrasonic mechanical dissection device by Covidien Surgical Solutions (Mansfield, Mass.). Critically, none of the devices are capable of radiofrequency output.

In contrast to the prior art, instruments designed in accordance with the principles of the present invention are electrosurgical, yet require no connection to an external electrosurgical generator. Moreover, the instruments of the present invention include on-board circuitry that enables the delivery of both DC and RF energy. Activation is manually controlled, through one or more fingers/hands, with built-in circuitry for initiating a pre-programmed module that monitors and controls the delivery of pulsed radiofrequency energy for a pre-determined amount of time.

The electrosurgical instruments of the present invention preferably include a power-conditioning module that allows the instrument to work either continuously or intermittently. During intermittent operation, the power-conditioning module can permit, for a limited duration, operation at peak power levels that are much higher than the average power possible in the continuous mode of operation.

Thus in addition to addressing a long felt need in the art, the electrosurgical instruments of the present invention offer improved clinical performance, better economic value, freedom of movement and enhanced mobility. Furthermore, as the instruments of the present invention require neither a maintenance schedule nor a large capital equipment outlay, they may be readily adopted at a minimized cost.

### SUMMARY OF THE INVENTION

The present invention is directed to electrosurgical instruments that are not only portable and self-powered but easily manipulated, modified and/or adapted for multiple divergent uses. More particularly, the present invention is directed to a novel electrosurgical instrument comprised of a portable energy source (such as a battery) and a hand-held power module in electrical communication, wherein the power module is provided with circuitry capable of supplying both RF energy suitable for electrosurgical procedures and power for causing resistive heating for thermal cautery. In a preferred embodiment, the instrument further includes a hand-held control element for activating and controlling the power module and a distal end electrode assembly for delivering both heat and energy into a soft tissue. The instrument components may comprise discrete, separable subassembly elements that can be readily and routinely exchanged, depending on the intended application and/or target tissue. Alternatively, one or more of the subassembly components may be integrated into a single instrument, e.g., a unitary power module and control element provided with a demountable battery and/or demountable active distal tip electrode.

In contrast to prior art instruments, the electrosurgical instruments of the present invention require no connection to an external power source, such as a medical electrosurgical generator; as such, they are free from cumbersome external electrical cords and the problems associated therewith.

The electrosurgical instruments of the present invention function by delivering electromagnetic energy to the tissue to be treated and can be monopolar, bipolar, or multipolar, with optional floating electrodes. They may be single use (disposable) or multi-use (reusable). Depending on the proposed application, they can operate with or without a patient-mounted return electrode (referred to in the art as a “return pad” or “return plate”). The electrosurgical instruments of the present invention can be used in dry or wet fields, in the presence of bodily fluids (such as blood saliva and more), electrically conductive or non-conductive fluids. They may be adapted for irrigation and or aspiration of liquids, gases or cryogenics, either external, remote or on board. Electrosurgical instruments of the present invention may also be operated in dual mode, i.e., delivering thermal energy through heating of a filament that also acts as an electrode when the instrument is used in electrosurgery mode.

Electrosurgical instruments designed in accordance with the principles of the present invention can be compatible with various image-guiding systems, like fluoroscopic, ultrasound and others. Introduction of the instruments to the target treatment site can be done directly (percutaneously) or with the aid of a guidance device such as a syringe, hypodermic needle, cannula, resectoscope or the like.

Electrosurgical instruments designed in accordance with the principles of the present invention constitute a true innovation in the medical device industry, and find utility in connection with a wide variety of medical procedures, both human and veterinary. By decreasing the number of electrical cords in the operating room, the new devices are safer both for the patient and the medical staff, more efficient, user-friendly, more cost effective, and can transform how many medical procedures will be performed. Finally, instruments based on the principles of this invention can function over wide frequency range: radiofrequency (100 kHz to 20 MHz), microwave, millimeter wave, infrared, optical and UV.

These and other objects are accomplished in the invention herein disclosed, It will be understood by those skilled in the art that one or more aspects of this invention can meet certain of the above objectives, while one or more other aspects can meet certain other objectives. Each objective may not apply equally, in all its respects, to every aspect of this invention. As such, the preceding and foregoing objects should be viewed in the alternative with respect to any one aspect of this invention.

The above-noted objects, aspects and features of the invention will become more fully apparent when the following detailed description is read in conjunction with the accompanying figures and/or examples. However, it is to be understood that both the foregoing summary of the invention and the following detailed description are of preferred embodiments and not restrictive of the invention or other alternate embodiments of the invention. Various modifications and applications may occur to those who are skilled in the art, without departing from the spirit and the scope of the invention, as described by the appended claims. Likewise, other objects, features, benefits and advantages of the present invention will be apparent from this summary and certain embodiments described below, and will be readily apparent to those skilled in the art having knowledge of electrode design. Such objects, features, benefits and advantages apparent from the above in conjunction with the accompanying examples, data, figures and all reasonable inferences to be drawn there-from are specifically incorporated herein.

### BRIEF DESCRIPTION OF THE FIGURES

Various aspects and applications of the present invention will become apparent to the skilled artisan upon consideration of the brief description of the figures and the detailed description of the present invention and its preferred embodiments that follows:

FIG. 1 is a distal perspective exploded view of a portable electrosurgical instrument constructed in accordance with the principles of this invention.

FIG. 2 is a proximal perspective exploded view of the objects of FIG. 1.

FIG. 3 is a proximal perspective view of the power module for a portable electrosurgical instrument constructed in accordance with the principles of this invention.

FIG. 4 is a distal perspective view of the objects of FIG. 3.

FIG. 5 is a plan view of the objects of FIG. 3.

FIG. 6 is a side elevational view of the objects of FIG. 3.

FIG. 7 is a proximal end view of the objects of FIG. 3.

FIG. 8 is a distal end view of the objects of FIG. 3.

FIG. 9 is a proximal perspective view of the body of a portable electrosurgical instrument constructed in accordance with the principles of this invention.

FIG. 10 is a distal perspective view of the objects of FIG. 9.

FIG. 11 is a plan view of the objects of FIG. 9.

FIG. 12 is a side elevational view of the objects of FIG. 9.

FIG. 13 is a proximal end view of the objects of FIG. 9.

FIG. 14 is a distal end view of the objects of FIG. 9.

FIG. 15 is a proximal perspective view of an electrode for a portable electrosurgical instrument constructed in accordance with the principles of this invention.

FIG. 16 is a distal perspective view of the objects of FIG. 15.

FIG. 17 is a plan view of the objects of FIG. 15.

FIG. 18 is a side elevational view of the objects of FIG. 15.

FIG. 19 is a proximal end view of the objects of FIG. 15.

FIG. 20 is a distal end view of the objects of FIG. 15.

FIG. 21 is a perspective view of a portable electrosurgical instrument constructed in accordance with the principles of this invention.

FIG. 22 is a plan view of the objects of FIG. 22.

FIG. 23 is a side elevational view of the objects of FIG. 22.

FIG. 24 is a perspective view of the distal portion of a portable electrosurgical instrument constructed in accordance with the principles of this invention with a cutting blade tip mounted.

FIG. 25 depicts a portable electrosurgical instrument constructed in accordance with the principles of this invention with an optional return (dispersive) electrode connected.

### DETAILED DESCRIPTION OF THE PREFERRED EMBODIMENTS

Although any methods and materials similar or equivalent to those described herein can be used in the practice or testing of embodiments of the present invention, the preferred methods, devices, and materials are now described. However, before the present materials and methods are described, it is to be understood that this invention is not limited to the particular compositions, methodologies or protocols herein described, as these may vary in accordance with routine experimentation and optimization. It is also to be understood that the terminology used in the description is for the purpose of describing the particular versions or embodiments only, and is not intended to limit the scope of the present invention which will be limited only by the appended claims.

### Elements of the Present Invention

Unless otherwise defined, all technical and scientific terms used herein have the same meaning as commonly understood by one of ordinary skill in the art to which this invention belongs. In case of conflict, the present specification, including following definitions, will control.

The words “a”, “an”, and “the” as used herein mean “at least one” unless otherwise specifically indicated.

The term “proximal” refers to that end or portion which is situated closest to the user. For example, the proximal end of an instrument designed in accordance with the present invention would typically include the power module and handle.

The term “distal” refers to that end or portion situated farthest away from the user. For example, the distal end of an instrument designed in accordance with the present invention would typically include the conductive thermal treatment element or “active electrode assembly”.

In certain embodiments, the present invention makes reference to “fluid(s)”. As used herein, the term “fluid(s)” refers to liquid(s), either electrically conductive or non-conductive, and to gaseous material, or a combination of liquid(s) and gas(es). In the context of the present invention, the term “fluid” extends to body fluids, examples of which include, but not limited to, blood, peritoneal fluid, lymph fluid, pleural fluid, gastric fluid, bile, and urine.

The present invention makes reference to the ablation, coagulation and vaporization of tissue. As used herein, the term “tissue” refers to biological tissues, generally defined as a collection of interconnected cells that perform a similar function within an organism. Four basic types of tissue are found in the bodies of all animals, including the human body and lower multicellular organisms such as insects, including epithelium, connective tissue, muscle tissue, and nervous tissue. These tissues make up all the organs, structures and other body contents. The present invention is not limited in terms of the tissue to be treated but rather has broad application, including the resection and/or vaporization any target tissue with particular applicability to the ablation, destruction and removal of problematic joint tissues.

The instant invention has both human medical and veterinary applications. Accordingly, the terms “subject” and “patient” are used interchangeably herein to refer to the person or animal being treated or examined. Exemplary animals include house pets, farm animals, and zoo animals. In a preferred embodiment, the subject is a mammal.

In common terminology and as used herein, the term “electrode” may refer to one or more components of an electrosurgical instrument (such as an active electrode or a return electrode) or to the entire device, as in an “ablator electrode” or “cutting electrode”. Such electrosurgical instruments are often interchangeably referred to herein as “probes”, “devices” or “instruments”.

The present invention makes references to a portable energy source and hand-held power module in electrical communication. In the context of the present invention, the energy source preferably comprises a single-use or rechargeable battery. In a preferred embodiment, the battery is a nickel-cadmium or lithium-ion battery rechargeable via terminals that coordinate with a conventional charging cradle.

In the context of the present invention, the power module contains on-board control circuitry capable of supplying both (i) radio frequency (RF) power suitable for electrosurgical procedures and (ii) direct current or low frequency alternating current suitable for heating of a conductive thermal treatment element. Activation is manually controlled, through one or more fingers/hands, with built-in circuitry for initiating a pre-programmed module that monitors and controls the delivery of pulsed radiofrequency energy for a pre-determined amount of time. In this manner, hand-held electrosurgical instruments of the present invention may be free of external electrical cords and operate in the absence of a dedicated external power source such as an RF medical generator.

The power-conditioning module preferably allows the instrument to work either continuously or intermittently. During intermittent operation, the power-conditioning module can permit, for a limited duration, operation at peak power levels that are much higher than the average power possible in the continuous mode of operation.

The present invention makes reference to a hand-held control element for activating and controlling the device. In the context of the present invention, the control element may take the form of an independent and separate subassembly. Alternatively, the control element and power module may be integrated into a single hand piece unit that retains the portable energy source at its proximal end and coordinates with an integral or demountable active electrode assembly at its distal end.

The present invention makes reference to one or more “active electrodes” or “active elements”. As used herein, the term “active electrode” refers to one or more conductive elements formed from any suitable preferably spark-resistant metal material, such as stainless steel, nickel, titanium, molybdenum, tungsten, and the like as well as combinations thereof, connected, for example via wiring disposed within the control/handle portion of the instrument, to a power supply, for example, a rechargeable battery disposed within the power module component capable of generating the requisite electric field and thermal energy. Like the overall electrosurgical instrument, the size, shape and orientation of the active electrode itself and the active surface (i.e., ablating surface) defined thereby may routinely vary in accordance with the need in the art. It will be understood that certain geometries may be better suited to certain utilities. Accordingly, those skilled in the art may routinely select one shape over another in order to optimize performance for specific surgical procedures.

Electrosurgical instruments contemplated by the present invention may be fabricated in a variety of sizes and shapes to optimize performance in a particular surgical procedure. For instance, instruments configured for use in small joints may be highly miniaturized while those adapted for shoulder, knee and other large joint use may need to be larger to allow high rates of tissue removable. Likewise, electrosurgical instruments for use in arthroscopy, otolaryngology and similar fields may be produced with a rounded geometry, e.g., circular, cylindrical, elliptical and/or spherical, using turning and machining processes, while such geometries may not be suitable for other applications. Accordingly, the geometry (i.e., profile, perimeter, surface, area, etc.) may be square, rectangular, polygonal or have an irregular shape to suit a specific need.

In certain embodiments, the present invention makes reference to one or more “floating electrodes”. As used herein, the term “floating electrode” refers to one or more disconnected electrodes that may contact the surrounding conducting liquid and/or tissue. The electrical potential of such disconnected electrodes is “floating” and is determined by the size and position of the electrode, the tissue type and properties, and the presence or absence of bodily fluids or externally supplied fluid. “Floating” electrodes for electrosurgery are described in published Patent Application Nos. US 2005-0065510 and US 2005-023446, the contents of which are incorporated by reference herein in their entirety. In the context of the present invention, the “floating” electrode is preferably mounted in such a way that one portion of the electrode is in close proximity to the tip of the active electrode, in the region of high potential. Another portion of the floating electrode is preferably placed farther away, in a region of otherwise low potential. This region of low potential may be in contact with the fluid environment, in contact with tissue, or both.

In the context of the present invention, a floating electrode can generate and concentrate high power density in the vicinity of the active region, and results in more efficient liquid heating, steam bubble formation and bubble trapping in this region. This increases the probe efficiency, which, in turn, allows the surgeon to substantially decrease the applied RF power and thereby reduce the likelihood of patient burns and unintended local tissue injury. The probe may be operated so that the portion of the floating electrode in close proximity to the active electrode has sufficient current density to produce vaporization of the liquid and arcing so as to vaporize tissue. Alternatively, the probe may be operated so that the floating electrode contacts tissue, wherein those portions of the floating electrode in contact with the tissue have sufficient current density to thermally coagulate blood vessels and tissue. This is particularly useful for achieving hemostasis in vascular tissue, such as, for instance, that present when performing tonsillectomies.

In certain embodiments, the present invention makes reference to one or more “insulators” separating active and floating electrodes. As used herein, the term “insulator” refers to a non-conductive element formed from a suitable dielectric material, examples of which include, but are not limited to, alumina, zirconia, and high-temperature polymers.

In certain embodiments, the present invention makes reference to one or more “return electrodes”. As used herein, the term “return electrode” refers to one or more powered conductive elements to which current flows after passing from the active electrode(s) back to the general-purpose generator. This return electrode may take the form of a patient-mounted return pad. Alternatively, it may be located on the electrosurgical instrument. In either case, it is preferably formed from a suitable electrically conductive material, for example a metal material such as stainless steel, nickel, titanium, molybdenum, tungsten, aluminum and the like as well as combinations thereof.

### Utilities of the Present Invention

As noted above, the present invention is directed to electrosurgical instruments that are both portable and self-powered that employ high frequency voltage to cut, ablate and/or coagulate tissue, particularly joint tissue, in conductive fluid and semi-dry environments. However, as noted previously, the present invention is not restricted to one particular field of surgery but rather find utility in connection with a wide variety of application, from arthroscopics to reconstructive, cosmetic, oncological, ENT, urological, gynecological, and/or laparascopic procedures, as well as in the context of general open surgery.

Electrosurgical instruments designed in accordance with the principles of the present invention can be useful for a variety of medical, both human and veterinary, applications for cutting, cauterization, coagulation, evaporation, sculpting, shrinking, smoothing, lesion formation, among others, in various types of tissue. The instruments can be used in a variety of medical procedures, like minimally invasive or open surgery, cosmetic or dermatological, on the surface or inside the body.

To that end, the active area of the instrument (i.e., the distal tip) can take many shapes and forms, and can be configured to meet the needs of the specific procedure in such fields, for example, as dental, urological, dermatological, cardiology, ear nose & throat, treatment of blood vessels, treatment of tumors as well as others. For example, for accessing narrow structures like vertebral discs it may be desirable to use an elongated electrode of a narrow geometry, e.g., having a relatively flat profile. Thus, for the most part, choices in geometry constitute a design preference. Alternatively, it may be desirable to utilize a miniaturized bipolar electrode assembly such as described in U.S. Provisional Application Ser. No. 61/956,357 filed Jun. 6, 2013, the entire contents of which are incorporated by reference herein.

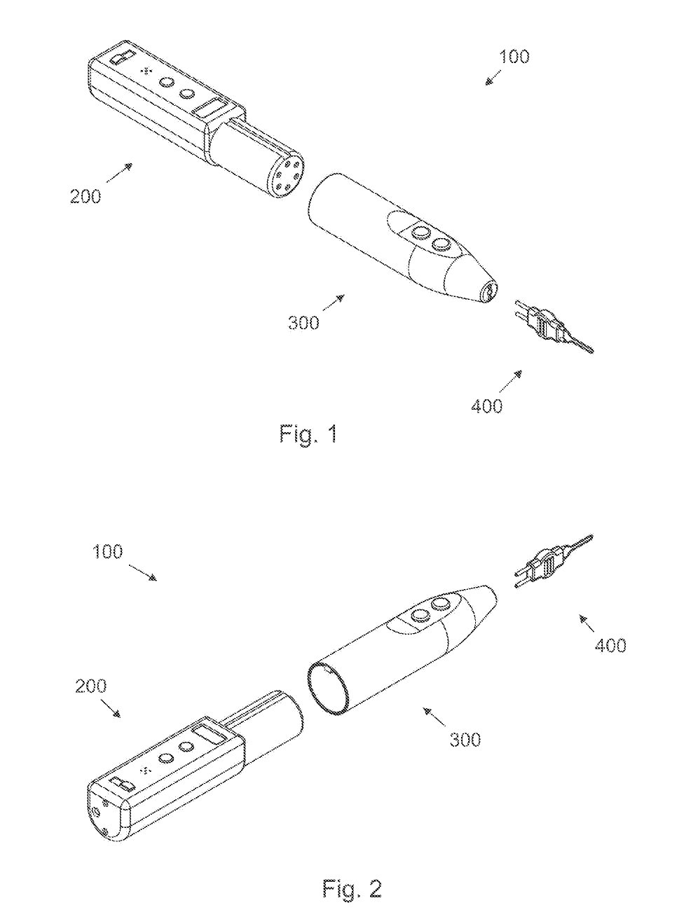

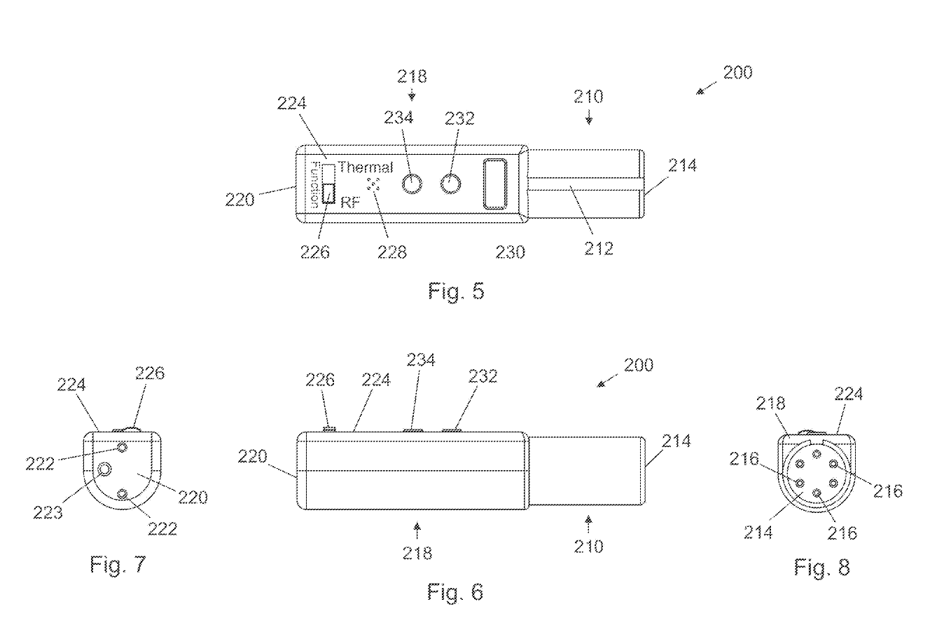

The instrument can be activated according to particular needs by using an activation control, and can be operated continuously or intermittently. Charging the energy storage module of the portable instrument between activations is possible, if needed, by placing the instrument in a charging cradle or by connecting it to another energy source. The energy storage element, or module (shown in FIGS. 2 to 6) can be for example a battery, rechargeable battery, high energy density capacitor, chemical or mechanical storage element, fuel cell, compressed gas among others.

As described previously the power-conditioning module will allow the instrument to work both continuously or intermittently. During intermittent operation the power-conditioning module will allow, for a limited duration, operation at peak power levels that are much higher then the average power possible in the continuous mode of operation.

While some embodiments of the present invention are designed to operate in dry or semi-dry environments, others utilize the endogenous fluid of a “wet field” environment to transmit current to target sites. Still others require the use of an exogenous irrigant. In certain embodiments, the “irrigant” (whether native or externally applied) is heated to the boiling point, whereby thermal tissue treatment arises through direct contact with either the boiling liquid itself or steam associated therewith. This thermal treatment may include desiccation to stop bleeding (hemostasis), and/or shrinking, denaturing, or enclosing of tissues for the purpose of volumetric reduction (as in the soft palate to reduce snoring) or to prevent aberrant growth of tissue, for instance, endometrial tissue or malignant tumors. However, the present invention is not particularly limited to the treatment of any one specific disease, body part or organ or the removal of any one specific type of tissue, the components and instruments of the present invention.

Liquids (either electrically conductive or non-conductive) and gaseous irrigants, either singly or in combination may also be advantageously applied to instruments for incremental vaporization of tissue. Normal saline solution may be used. Alternatively, the use of low-conductivity irrigants such as water or gaseous irrigants or a combination of the two allows increased control of the electrosurgical environment.

The electrosurgical instruments of the present invention may be used in conjunction with existing diagnostic and imaging technologies, for example imaging systems including, but not limited to, MRI, CT, PET, x-ray, fluoroscopic, thermographic, photo-acoustic, ultrasonic and gamma camera and ultrasound systems. Such imaging technology may be used to monitor the introduction and operation of the instruments of the present invention. For example, existing imaging systems may be used to determine location of target tissue, to confirm accuracy of instrument positioning, to assess the degree of tissue vaporization (e.g., sufficiency of tissue removal), to determine if subsequent procedures are required (e.g., thermal treatment such as coagulation and/or cauterization of tissue adjacent to the target tissue and/or surgical site), and to assist in the traumatic removal of the instrument.

### Illustrative Embodiments of the Present Invention

Hereinafter, the present invention is described in more detail by reference to the exemplary embodiments. However, the following examples only illustrate aspects of the invention and in no way are intended to limit the scope of the present invention. As such, embodiments similar or equivalent to those described herein can be used in the practice or testing of the present invention.

FIGS. 1 and 2 depict an electrosurgical instrument constructed in accordance with the principles of this invention. In a preferred embodiment, instrument 100 is an assembly formed from the assembly of a battery/power module 200 to a control handle 300 and electrode tip assembly 400.

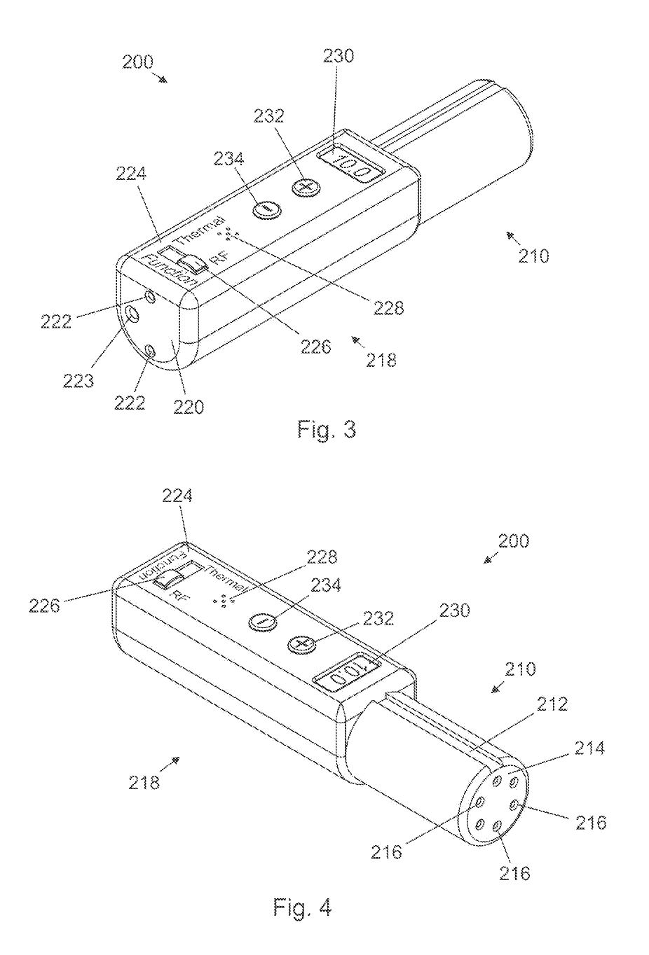

FIGS. 3 through 8 depict an illustrative battery/power module 200 which contains a battery and circuitry for supplying to handle 300 and, by wiring means therein, to electrode tip assembly 400 RF energy suitable for electrosurgical procedures, as well as power for causing resistive heating of electrode tip assembly 400 for thermal cautery. Module 200 includes a cylindrical distal portion 210 with an axial alignment channel 212, a distal-most surface 214 having formed therein recesses 216 for accepting pins of an electrical connector. Module 200 includes a proximal portion 218 having a proximal-most surface 220 having formed therein recesses 222 for accepting pins of an electrical connector, and thereby allow electrical connection to a charging cradle. Proximal-most surface 220 further includes a connection 223 for an optional return electrode or other external element. Proximal portion 218 has a top surface 224 provided with a switch 226 optionally labeled “Function” with a first position optionally labeled “Thermal” and a second position optionally labeled “RF”. Top surface 224 has formed therein a speaker element 228 for transmitting an audio tone, a display element 230 for displaying numerical data such as a power level value, and first and second “volume” buttons 232 and 234, said buttons 232 and 234 having “+” and “−” indicia formed thereon respectively wherein the “+” button functions to increase power and/or frequency and the “−” button serves to decrease it.

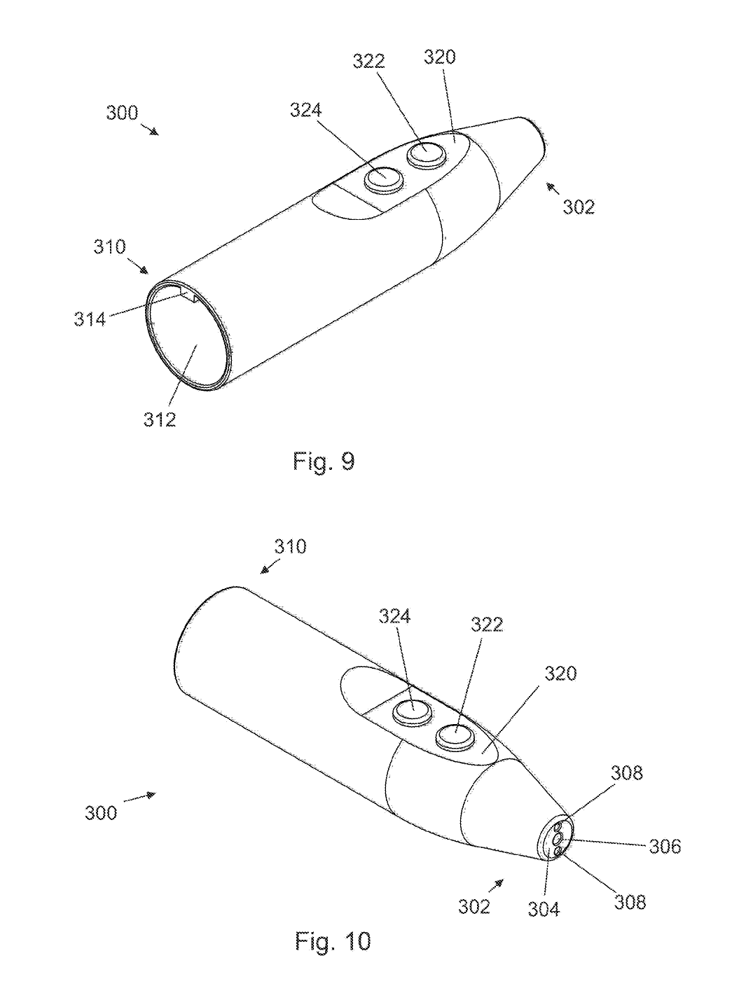

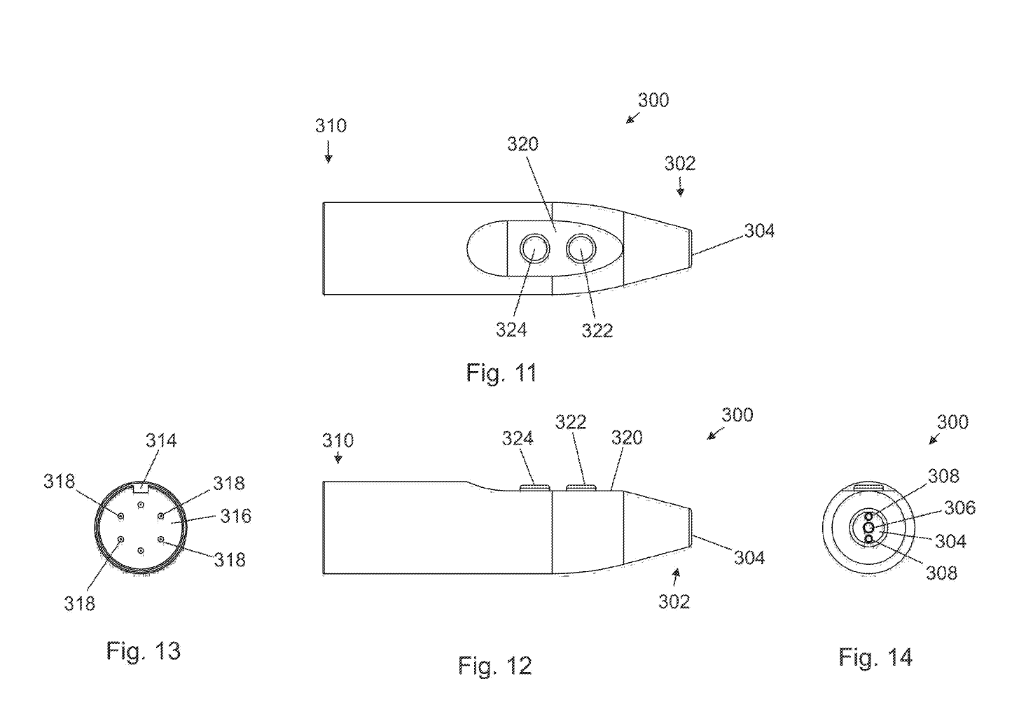

FIGS. 9 through 14 depict control handle 300 having a distal end 302 with a distal-most surface 304 having formed therein first central recess 306 and second recesses 308, recesses 306 and 308 being configured as receptacles to retain and transmit electrical energy to conductive elements removably inserted therein. Control handle 300 has a proximal end 310 having formed therein recess 312 with alignment key 314, wherein recess 312 and key 314 are configured to accept cylindrical distal portion 210 of module 200, with key 314 slidably mating with axial alignment channel 212. Recess 312 has a distal-most surface 316 from which protrude electrical connector pins 318 which are configured to mate with recesses 216 to form an electrical connector pair. Control handle 300 further has a top surface 320 from which protrude first button 322 and second button 324.

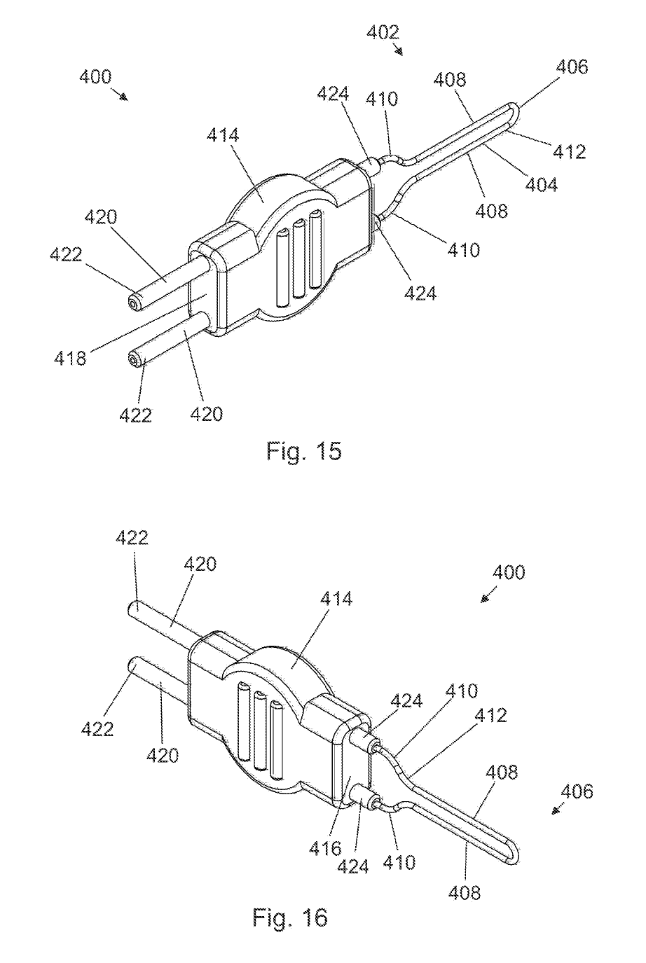

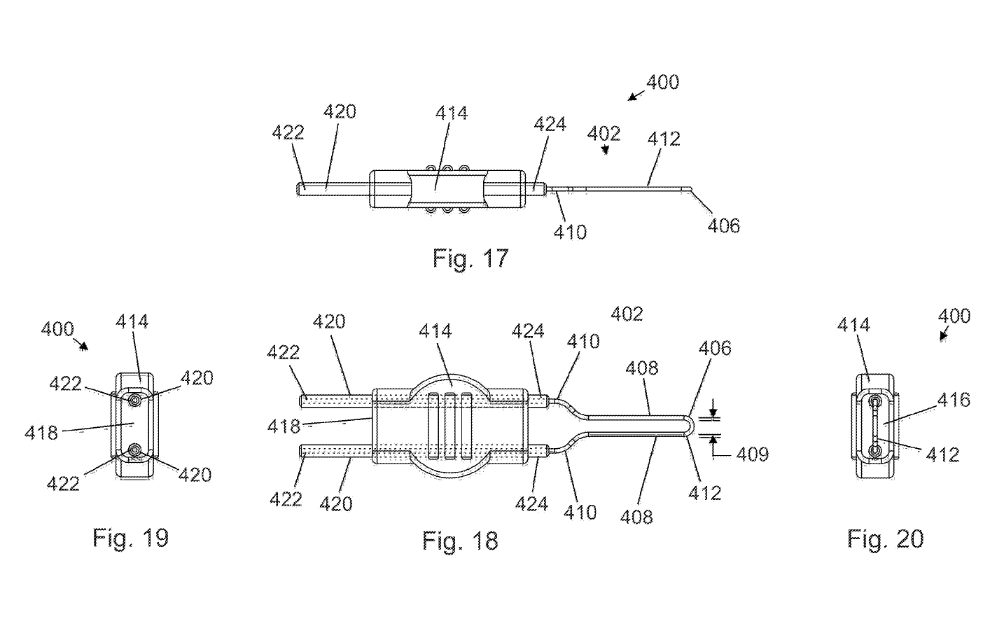

FIGS. 15 through 20 depict electrode assembly 400 formed of elongate conductive elements 420, polymeric grip portion 414, and distal electrode element 402. Proximal portions 422 of conductive elements 420 protrude from proximal face 418 of grip portion 414, portions 422 being configured to allow removable mounting of electrode assembly 400 to distal end 302 of handle 300 using recesses 308 in distal-most surface 304 of handle 300, electrode assembly 400 being thereby electrically connected to circuitry within control handle 300. Electrode element 402 is formed from a contoured elongate element 412 having its proximal ends 410 mounted to distal ends 424 of elongate conductive elements 402 which protrude beyond distal-most surface 416 of grip portion 414. Electrode element 402 has parallel mid-portions 408 spaced distance 409 apart and connected at their distal ends by distal portion 406 of element 402.

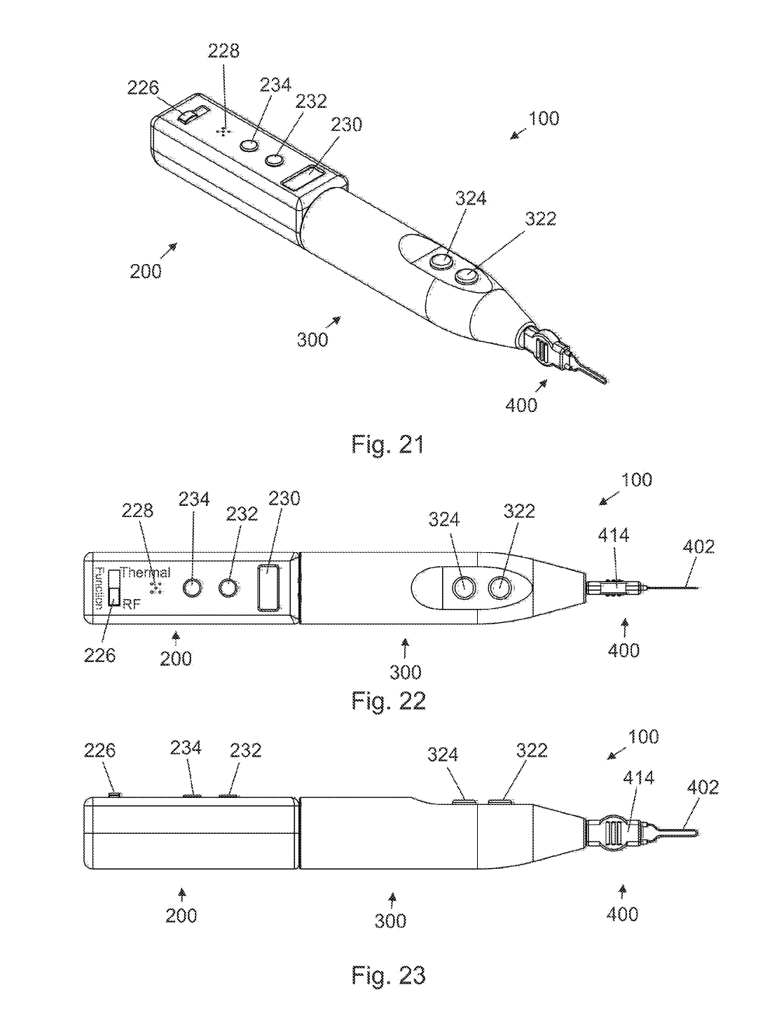

FIGS. 21 through 23 depict a portable electrosurgical instrument 100 constructed in accordance with the principles of this invention, assemble from components 200, 300, and 400. When switch 226 is placed in the “Thermal” position, pressing first button 322 causes DC power to be supplied to electrode assembly 400. In a preferred embodiment, pressing second button 324 causes a pulse of DC current of predetermined duration to assembly 400. When switch 226 is placed in the RF position, the RF power level may be set to a desired value on display 230 using increment and decrement buttons 232 and 234. Thereafter, depressing first button 322 causes RF energy of the selected power level and having a first waveform to electrode assembly 400. An audible tone may be emitted via speaker element 228 while first button 322 is depressed. In a similar manner, a second power level for a second waveform may be supplied to electrode assembly 400 when second button 324 is depressed.

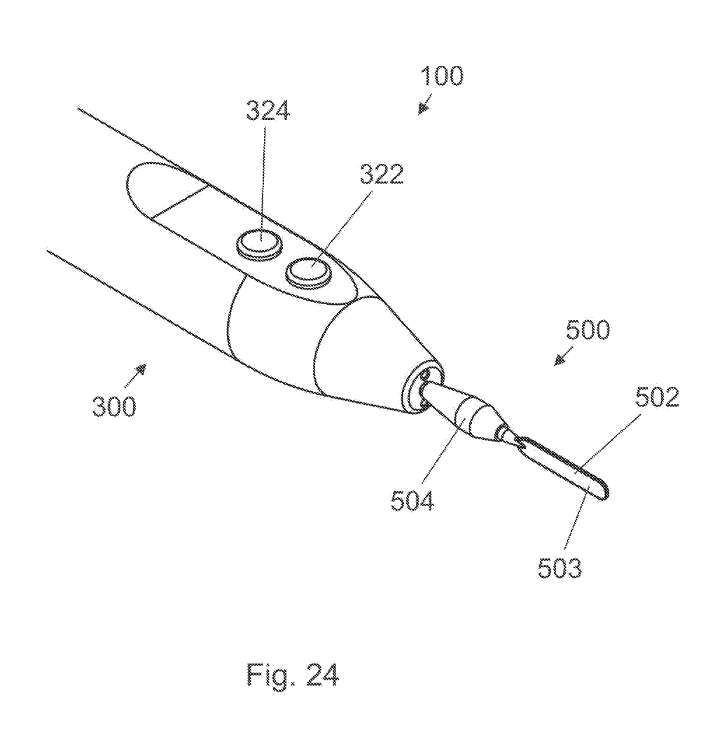

Portable electrosurgical instrument 100 may also be used with a blade electrode as depicted in FIG. 24. Blade electrode assembly 500 has a metallic element 502 having a distal portion 503 formed to a flat blade and a proximal portion mounted in jack 306 (FIG. 10), the mid-portion therebetween being covered by polymeric hub 504.

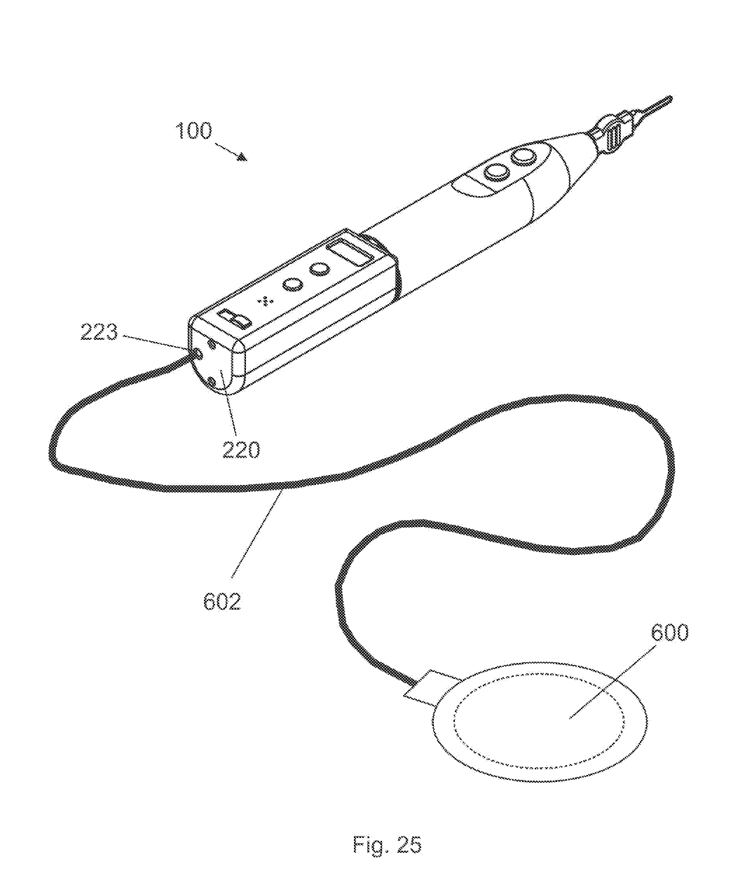

Portable electrosurgical instruments constructed in accordance with the principles of this invention may optionally be used with a return electrode. FIG. 25 depicts portable electrosurgical instrument 100 connected by cable 602 to return electrode 600, return electrode 600 being in the form of a dispersive pad. Because the average power levels at which instrument 100 operate are low, return electrode 600 may have a smaller area than conventional adult or pediatric return pads. In other embodiments, return electrode 600 is not used, but an external element not connected to the patient is connected to connection 223, the external element formed in a manner to produce efficient capacitive coupling.

In contrast to prior art, the instruments designed in accordance with the principles of this invention are electrosurgical, yet require no connection to an external electrosurgical generator. Activation is controlled by finger/hand, and there is built in circuitry for initiating preprogrammed means to monitor and control the delivery of pulsed radiofrequency energy for predetermined periods of time as well as thermal energy.

Referring to FIGS. 1 and 2, the preferred embodiment instrument 100 is depicted as an assembly of three discrete subassemblies. In other embodiments, battery/power module 200 and handle 300 may be combined in a single assembly or handle 300 and electrode tip assembly 400 may be combined in a single assembly, or all three subassemblies may be combined in a single assembly. Alternatively, module 200 and handle 300 may be combined in a single assembly with the battery portion of module 200 being replaced by an externally mounted battery which may be separated from instrument 100 for recharging. Also, while instrument 100 has capability for both radio frequency and thermal treatment of tissue, in other embodiments only radio frequency power is supplied for treatment. Instrument 100 is a monopolar device. Other embodiments are anticipated which are able to operate in either a monopolar or bipolar mode. Any electrosurgical device that supplies RF energy for the vaporization or thermal treatment of tissue that is not connected to an external RF generator falls within the scope of this invention.

### INDUSTRIAL APPLICABILITY

Electrosurgical instruments designed based on the principles of the present invention offer improved clinical performance, better economic value, freedom of movement and enhanced mobility, reduced overhead costs since with no maintenance schedule, and are easy to adopt with no large capital equipment outlay.

All patents and publications mentioned herein are incorporated by reference in their entirety. Nothing herein is to be construed as an admission that the invention is not entitled to antedate such disclosure by virtue of prior invention.

While the invention has been described in detail and with reference to specific embodiments thereof, it is to be understood that the foregoing description is exemplary and explanatory in nature and is intended to illustrate the invention and its preferred embodiments. Through routine experimentation, one skilled in the art will readily recognize that various changes and modifications can be made therein without departing from the spirit and scope of the invention. Such other advantages and features will become apparent from the claims filed hereafter, with the scope of such claims to be determined by their reasonable equivalents, as would be understood by those skilled in the art. Thus, the invention is defined not by the above description, but by the following claims and their equivalents.

What is claimed:

1. A hand-held electrosurgical instrument comprising a power module and portable energy source in electrical communication, wherein said power module contains control circuitry capable of supplying both (i) radio frequency (RF) power suitable for electrosurgical procedures and (ii) direct current or low frequency alternating current suitable for heating of a conductive thermal treatment element, further wherein said hand-held electrosurgical device is free of external electrical cords and operates in the absence of an external power source.

2. The device of claim 1 wherein said portable energy source comprises a battery that is demountable from the power module.

3. The device of claim 2, wherein said demountable battery comprises a rechargeable battery adapted for use with a conventional charging cradle.

4. The device of claim 1 wherein said device is comprised of a series of subassemblies.

5. The device of claim 4, wherein the first of said series of subassemblies comprises said energy source and power module.

6. The device of claim 5, wherein the second of said subassemblies comprises a hand-held control element for activating and controlling said first subassembly.

7. The device of claim 6, wherein the third of said subassemblies comprises a conductive thermal treatment element.

8. The device of claim 4, wherein one or more of said subassemblies is demountable.

9. The device of claim 8, wherein said third subassembly is demountable.

10. The device of claim 9, wherein said third subassembly comprises a demountable electrode assembly.

11. The device of claim 10, wherein said demountable electrode assembly comprises a loop electrode.

12. The device of claim 10, wherein said demountable electrode assembly comprises an active electrode, a floating electrode, and an insulator separating said active and floating electrodes.

13. The device of claim 4, wherein one or more of said subassemblies are integrated.

14. The device of claim 13, wherein said first and second subassemblies are combined into a single hand piece assembly.

15. The device of claim 1, wherein said instrument is coupled with a means for supplying fluid to the region of the instrument distal end so as to irrigate a target tissue site.

16. The device of claim 1, wherein said instrument is coupled with a means for aspirating fluid and ablation products from the region of the instrument distal end.

17. The device of claim 1, wherein said control circuitry allows the instrument to work either continuously or intermittently.

18. The device of claim 1, wherein said control circuitry allows for delivery of pulsed radiofrequency energy for a pre-determined amount of time.
